SEQUENCE LISTING (19 sequences)

SEQ ID NO: 1 - DNA, 1137 bp, Homo sapiens
  TAP-38 gene sequence
atgagcggcagagtcggcgatctgagccccaggcagaaggaggcattggccaagccagaagcttcgacctgcagaagtcggaggccatgctccggaagcatgtggagttccgaaagcaaaaggacattgacaacatcattagcatggcagcctccagaggtgatccaacagtatctgtcactggatgccaagggtctgctgttctcagcctccaaacaggacctgctgaggaccaagatgctggatgccaagggtctgctgttctcagcctccaaacaggacctgctgaggaccaagatgcgggagtgtgagctgcttctgcaagagtgtgcccaccagaccacaaagttggggaggaaggtggagaccatcaccataatttatgactgcgaggggcttggcctcaagcatctctggaagcctgctgtggaggcctatggagagtttctctgcatgtttgaggaaaattatcccgaaacactgaagcgtctttttgttgttaaagcccccaaactgtttcctgtggcctataacctcatcaaacccttcctgagtgaggacactcgtaagaagatcatggtcctgggagcaaattggaaggaggttttactgaaacatatcagccctgaccaggtgcctgtggagtatgggggcgccatgactgaccctgatggaaaccccaagtgcaaatccaagatcaactacgggggtgacatccccaggaagtattatgtgcgagaccaggtgaaacagcagtatgaacacagcgtgcagatttcccgtggctcctcccaccaagtggagtatgagatcctcttccctggctgtgtcctcaggtggcagtttatgtcagatggagcggatgttggttttgggattttcctgaagaccaagatgggagagaggcagcgggcaggggagatgacagaggtgctgcccaaccagaggtacaactcccacctggtccctgaagatgggaccctcacctgcagtgatcctggcatctatgtcctgcggtttgacaacacctacagcttcattcatgccaagaaggtcaatttcactgtggaggtcctgcttccagacaaagcctcagaagagaagatgaaacagctgggggcaggcaccccgaaataa

SEQ ID NO: 2 - protein, 378 aa, Homo sapiens
  PEPTIDE: TAP-38 polypeptide
MSGRVGDLSPRQKEALAKPEASTCRSRRPCSGSMWSSESKRTLTTSLAWQPPEVIQQYLSGGMCGYDLDGCPVWYDIIGPKDAKGLLFSASKQDLLRTKMRECELLLQECAHQTTKLGRKVETITIIYDCEGLGLKHLWKPAVEAYGEFLCMFEENYPETLKRLFVVKAPKLFPVAYNLIKPFLSEDTRKKIMVLGANYKEVLLKHISPDQVPVEYGGTMTDPDGNPKCKSKINYGGDIPRKYYVRDQVKQQYEHSVQISRGSSHQVEYEILFPGCVLRWQFMSDGADVGFGIFLKTKMGERQRAGEMTEVLPNQRYNSHLVPEDGTLTCSDPGIYVLRFDNTYSFIHAKKVNFTVEVLLPDKASEEKMKQLGAGTPK

SEQ ID NO: 3 - DNA, 1212 bp, Homo sapiens
  TAP-46 gene sequence
atgagcggcagagtcggcgatctgagccccaggcagaaggaggcattggccaagtttcgggagaatgtccaggatgtgctgccggccctgccgaatccagatgactattttctcctgcgttggctccgagccagaagcttcgacctgcagaagtcggaggccatgctccggaagcatgtggagttccgaaagcaaaaggacattgacaacatcattagctggcagcctccagaggtgatccaacagtatctgtcactggatgccaagggtctgctgttctcagcctccaaacaggacctgctgaggaccaagatgctggatgccaagggtctgctgttctcagcctccaaacaggacctgctgaggaccaagatgcgggagtgtgagctgcttctgcaagagtgtgcccaccagaccacaaagttggggaggaaggtggagaccatcaccataatttatgactgcgaggggcttggcctcaagcatctctggaagcctgctgtggaggcctatggagagtttctctgcatgtttgaggaaaattatcccgaaacactgaagcgtctttttgttgttaaagcccccaaactgtttcctgtggcctataacctcatcaaacccttcctgagtgaggacactcgtaagaagatcatggtcctgggagcaaattggaaggaggttttactgaaacatatcagccctgaccaggtgcctgtggagtatgggggcgccatgactgaccctgatggaaaccccaagtgcaaatccaagatcaactacgggggtgacatccccaggaagtattatgtgcgagaccaggtgaaacagcagtatgaacacagcgtgcagatttcccgtggctcctcccaccaagtggagtatgagatcctcttccctggctgtgtcctcaggtggcagtttatgtcagatggagcggatgttggttttgggattttcctgaagaccaagatgggagagaggcagcgggcaggggagatgacagaggtgctgcccaaccagaggtacaactcccacctggtccctgaagatgggaccctcacctgcagtgatcctggcatctatgtcctgcggtttgacaacacctacagcttcattcatgccaagaaggtcaatttcactgtggaggtcctgcttccagacaaagcctcagaagagaagatgaaacagctgggggcaggcaccccgaaataa

SEQ ID NO: 4 - protein, 403 aa, Homo sapiens
  PEPTIDE: TAP-46 polypeptide
MSGRVGDLSPRQKEALAKFRENVQDVLPALPNPDDYFLLRWLRARSFDLQKSEAMLRKHVEFRKQKDIDNIISWQPPEVIQQYLSGGMCGYDLDGCPVWYDIIGPLDAKGLLFSASKQDLLRTKMRECELLLQECAHQTTKLGRKVETITIIYDCEGLGLKHLWKPAVEAYGEFLCMFEENYPETLKRLFVVKAPKLFPVAYNLIKPFLSEDTRKKIMVLGANWKEVLLKHISPDQVPVEYGGTMTDPDGNPKCKSKINYGGDIPRKYYVRDQVKQQYEHSVQISRGSSHQVEYEILFPGCVLRWQFMSDGADVGFGIFLKTKMGERQRAGEMTEVLPNQRYNSHLVPEDGTLTCSDPGIYVLRFDNTYSFIHAKKVNFTVEVLLPDKASEEKMKQLGAGTPK

SEQ ID NO: 5 - DNA, 21 bp, artificial sequence
  primer_bind: TAP-38 sense oligonucleotide
atgagcggcagagtcggcgat

SEQ ID NO: 6 - DNA, 23 bp, artificial sequence
  primer_bind: TAP-38 antisense oligonucleotide
ttatttcggggtgcctgccccca

SEQ ID NO: 7 - DNA, 57 bp, artificial sequence
  primer_bind: TAP-38 sense oligonucleotide encoding HA-tag
cgcgaattcatgtatgatgttcctgattatgctagcctcagcggcagagtcggcgat

SEQ ID NO: 8 - protein, 16 aa, Homo sapiens
  PEPTIDE: 16 amino acids from the c-terminus of TAP attached to keyhole limpet hemocyanin
KASEEKMKQLGAGTPK

SEQ ID NO: 9 - DNA, 27 bp, artificial sequence
  CDS: sequence for an HA tag
tatgatgttcctgattatgctagcctc

SEQ ID NO: 10 - DNA, 59 bp, artificial sequence
  primer_bind: sense primer for the deletion mutant TAP-882
cgcgaattcatgtatgatgttcctgattatgctagcctcctgctgttctcagcctccaa

SEQ ID NO: 11 - DNA, 59 bp, artificial sequence
  primer_bind: sense primer for the deletion mutant TAP-681
cgcgaattcatgtatgatgttcctgattatgctagcctctttgaggaaaattatcccga

SEQ ID NO: 12 - DNA, 59 bp, artificial sequence
  primer_bind: sense primer for the deletion mutant TAP-456
cgcgaattcatgtatgatgttcctgattatgctagcctcaagtgcaaatccaagatcaa

SEQ ID NO: 13 - DNA, 21 bp, artificial sequence
  primer_bind: antisense primer for the TAP deletion mutants
tttcggggtgcctgcccccag

SEQ ID NO: 14 - DNA, 882 bp, Homo sapiens
  deletion mutant TAP-882
ctgctgttctcagcctccaaacaggacctgctgaggaccaagatgcgggagtgtgagctgcttctgcaagagtgtgcccaccagaccacaaagttggggaggaaggtggagaccatcaccataatttatgactgcgaggggcttggcctcaagcatctctggaagcctgctgtggaggcctatggagagtttctctgcatgtttgaggaaaattatcccgaaacactgaagcgtctttttgttgttaaagcccccaaactgtttcctgtggcctataacctcatcaaacccttcctgagtgaggacactcgtaagaagatcatggtcctgggagcaaattggaaggaggttttactgaaacatatcagccctgaccaggtgcctgtggagtatgggggcaccatgactgaccctgatggaaaccccaagtgcaaatccaagatcaactacgggggtgacatccccaggaagtattatgtgcgagaccaggtgaaacagcagtatgaacacagcgtgcagatttcccgtggctcctcccaccaagtggagtatgagatcctcttccctggctgtgtcctcaggtggcagtttatgtcagatggagcggatgttggttttgggattttcctgaagaccaagatgggagagaggcagcgggcaggggagatgacagaggtgctgcccaaccagaggtacaactcccacctggtccctgaagatgggaccctcacctgcagtgatcctggcatctatgtcctgcggtttgacaacacctacagcttcattcatgccaagaaggtcaatttcactgtggaggtcctgcttccagacaaagcctcagaagagaagatgaaacagctgggggcaggcaccccgaaataa

SEQ ID NO: 15 - protein, 293 aa, Homo sapiens
  PEPTIDE: deletion mutant TAP-882
LLFSASKQDLLRTKMRECELLLQECAHQTTKLGRKVETITIIYDCEGLGLKHLWKPAVEAYGEFLCMFEENYPETLKRLFVVKAPKLFPVAYNLIKPFLSEDTRKKIMVLGANWKEVLLKHISPDQVPVEYGGTMTDPDGNPKCKSKINYGGDIPRKYYVRDQVKQQYEHSVQISRGSSHQVEYEILFPGCVLRWQFMSDGADVGFGIFLKTKMGERQRAGEMTEVLPNQRYNSHLVPEDGTLTCSDPGIYVLRFDNTYSFIHAKKVNFTVEVLLPDKASEEKMKQLGAGTPK

SEQ ID NO: 16 - DNA, 681 bp, Homo sapiens
  deletion mutant TAP-681
tttgaggaaaattatcccgaaacactgaagcgtctttttgttgttaaagcccccaaactgtttcctgtggcctataacctcatcaaacccttcctgagtgaggacactcgtaagaagatcatggtcctgggagcaaattggaaggaggttttactgaaacatatcagccctgaccaggtgcctgtggagtatgggggcaccatgactgaccctgatggaaaccccaagtgcaaatccaagatcaactacgggggtgacatccccaggaagtattatgtgcgagaccaggtgaaacagcagtatgaacacagcgtgcagatttcccgtggctcctcccaccaagtggagtatgagatcctcttccctggctgtgtcctcaggtggcagtttatgtcagatggagcggatgttggttttgggattttcctgaagaccaagatgggagagaggcagcgggcaggggagatgacagaggtgctgcccaaccagaggtacaactcccacctggtccctgaagatgggaccctcacctgcagtgatcctggcatctatgtcctgcggtttgacaacacctacagcttcattcatgccaagaaggtcaatttcactgtggaggtcctgcttccagacaaagcctcagaagagaagatgaaacagctgggggcaggcaccccgaaataa

SEQ ID NO: 17 - protein, 226 aa, Homo sapiens
  PEPTIDE: deletion mutant TAP-681
FEENYPETLKRLFVVKAPKLFPVAYNLIKPFLSEDTRKKIMVLGANWKEVLLKHISPDQVPVEYGGTMTDPDGNPKCKSKINYGGDIPRKYYVRDQVKQQYEHSVQISRGSSHQVEYEILFPGCVLRWQFMSDGADVGFGIFLKTKMGERQRAGEMTEVLPNQRYNSHLVPEDGTLTCSDPGIYVLRFDNTYSFIHAKKVNFTVEVLLPDKASEEKMKQLGAGTPK

SEQ ID NO: 18 - DNA, 456 bp, Homo sapiens
  deletion mutant TAP-456
aagtgcaaatccaagatcaactacgggggtgacatccccaggaagtattatgtgcgagaccaggtgaaacagcagtatgaacacagcgtgcagatttcccgtggctcctcccaccaagtggagtatgagatcctcttccctggctgtgtcctcaggtggcagtttatgtcagatggagcggatgttggttttgggattttcctgaagaccaagatgggagagaggcagcgggcaggggagatgacagaggtgctgcccaaccagaggtacaactcccacctggtccctgaagatgggaccctcacctgcagtgatcctggcatctatgtcctgcggtttgacaacacctacagcttcattcatgccaagaaggtcaatttcactgtggaggtcctgcttccagacaaagcctcagaagagaagatgaaacagctgggggcaggcaccccgaaataa

SEQ ID NO: 19 - protein, 151 aa, Homo sapiens
  PEPTIDE: deletion mutant TAP-456
KCKSKINYGGDIPRKYYVRDQVKQQYEHSVQISRGSSHQVEYEILFPGCVLRWQFMSDGADVGFGIFLKTKMGERQRAGEMTEVLPNQRYNSHLVPEDGTLTCSDPGIYVLRFDNTYSFIHAKKVNFTVEVLLPDKASEEKMKQLGAGTPK

(19) United States
(12) Patent Application Publication
(10) Pub. No.: US 2004/0152883 A1
(43) Pub. Date: Aug. 5, 2004

(54) Tocopherol associated protein and uses thereof

(76) Inventors: Bob G. Sanders, Austin, TX (US); Kimberly Kline, Austin, TX (US); Weiping Yu, Austin, TX (US); Hui Liu, Austin, TX (US); Feras M. Hantash, Dana Point, CA (US)

Correspondence Address: Dr. Benjamin Adler, ADLER & ASSOCIATES, 8011 Candle Lane, Houston, TX, 77071, US

(21) Appl. No.: 10/696,699

(22) Filed: Oct. 29, 2003

Related U.S. Application Data
(63) Continuation-in-part of application No. 10/419,629, filed on Apr. 21, 2003.
(60) Provisional application No. 60/373,870, filed on Apr. 19, 2002.

(51) Int. Cl. C07H 21/04; C12N 9/02; C12N 9/10

(52) U.S. Cl. 536/23.2; 435/193; 435/320.1; 435/325; 435/69.1; 435/189

(57) ABSTRACT

The present invention discloses cloning of tocopherol associated protein TAP-38 and deletion mutants of TAP-46. TAP-38, which shares homology with the previously identified TAP-46, enhances the apoptotic inducing properties of tocopherol based compounds, and blockage of TAP-38 or TAP-46 reduces the effectiveness of tocopherol based compounds. Thus, delivery of TAP-38 and TAP-46 cDNA by aerosol liposome/transfection/infection methods, separately or in combination with tocopherol-based apoptosis-inducing agents as well as with other chemotherapeutic agents, would be useful for treatment and prevention of cellular proliferative diseases and disorders.

### CROSS-REFERENCE TO RELATED APPLICATION

This continuation-in-part application claims benefit of patent application Ser. No. 10/419,629, filed Apr. 21, 2003, which claims benefit of provisional patent application 60/373,870, filed Apr. 19, 2002, now abandoned.

### BACKGROUND OF THE INVENTION

1. Field of the Invention

The present invention relates generally to the fields of molecular genetics and cancer biology. More specifically, the present invention relates to cDNA cloning and nucleotide sequencing of a novel tocopherol associated proteins from human normal and breast cancer cells. These proteins are relevant to the ability of novel tocopherol compounds to inhibit DNA synthesis and induce apoptosis in cancer cells.

2. Description of the Related Art

The regulatory controls of cell proliferation and cell death (apoptosis) are extremely complex and involve multiple intracellular signaling pathways and multiple interacting gene products. Cancer cells may exhibit multiple defects in normal regulatory controls of cell proliferation, such as enhanced expression of genes, which allow them to increase in number. In addition to enhanced expression of genes related to cell growth, cancer cells down-regulate genes and their products that control apoptotic signals, resulting in the accumulation and potential metastasis of life threatening cancer cells. Thus, combinations of unregulated cell proliferation and suppression of cell-death inducing signaling pathways give cancer cells both growth and survival advantages.

Genes involved in apoptosis can be either pro-apoptotic or anti-apoptotic, and the dynamic balance between them determines whether a cell lives or dies. Cancer cells, in order to survive and increase their numbers, undergo a series of mutational events over time that remove regulatory controls that give them the ability to grow unchecked and survive even in the presence of pro-apoptotic signals, and develop attributes that permit them to escape detection and removal by the immune response defense system.

A wide variety of pathological cell proliferative conditions exist for which novel therapeutic strategies and agents are needed to provide therapeutic benefits. These pathological conditions may occur in almost all cell types capable of abnormal cell proliferation or abnormal responsiveness to cell death signals. Among the cell types that exhibit pathological or abnormal growth and death characteristics are fibroblasts, vascular endothelial cells, and epithelial cells. Thus, novel methods are needed to treat local or disseminated pathological conditions in all or almost all organ and tissue systems of individuals.

Most cancers, whether they are male specific (such as prostate or testicular), female specific (such as breast, ovarian or cervical) or whether they affect males and females equally (such as liver, skin or lung), undergo increased genetic lesions, and epigenetic events over time, and eventually become highly metastatic and difficult to treat. Surgical removal of localized cancers has proven effective only when the cancer has not spread beyond the primary lesion. Once the cancer has spread to other tissues and organs, the surgical procedures must be supplemented with other more specific procedures to eradicate the diseased or malignant cells. Most of the commonly utilized supplementary procedures for treating diseased or malignant cells such as chemotherapy or radiation are not localized to the tumor cells and, although they have a proportionally greater destructive effect on malignant cells, often affect normal cells to some extent.

Some natural vitamin E compounds and derivatives of vitamin E have been used as pro-apoptotic and DNA synthesis-inhibiting agents. Structurally, vitamin E is composed of a chromanol head and an alkyl side chain. There are eight major naturally occurring forms of vitamin E: alpha (α), beta (β), gamma (γ) and delta (δ) tocopherols and α, β, γ, and δ tocotrienols. Tocopherols differ from tocotrienols in that they have a saturated phytyl side chain rather than an unsaturated isoprenyl side chain. The four forms of tocopherols and tocotrienols differ in the number of methyl groups on the chromanol head (α has three, β and γ have two and δ has one).

RRR-α-tocopheryl succinate is a derivative of RRR-α-tocopherol that has been structurally modified via an ester linkage to contain a succinyl moiety instead of a hydroxyl moiety at the 6-position of the chroman head. This ester linked succinate moiety of RRR-α-tocopherol has been the most potent form of vitamin E affecting apoptosis and inhibiting DNA synthesis. This form of vitamin E induces tumor cells to undergo apoptosis, while having no apoptosis-inducing effects on normal cells. The succinated form of vitamin E is effective as an anticancer agent as an intact agent; however, cellular and tissue esterases that can cleave the succinate moiety, thereby converting the succinate form of RRR-α-tocopherol to the free RRR-α-tocopherol, render this compound ineffective as an anticancer agent. RRR-α-tocopherol exhibits neither anti-proliferative nor pro-apoptotic biological activity in cells of epithelial or immune origin. Attachment of the succinate moiety to the C-6 carbon on the chromonal ring of RRR-α-tocopherol via an ether linkage provides stable tocopherol based apoptosis-inducing compounds that cannot be rendered ineffective since cells do not have etherases to clip off the succinate moiety.

To understand the mechanisms of action of tocopherols and tocotrienols as anticancer agents requires an understanding of their binding and their inter- and intra-cellular transport via proteins that specifically interact with these compounds. It is well established that very low density lipoproteins (VLDLs) are loaded with RRR-α-tocopherol in the liver allowing for the entrance of RRR-α-tocopherol into circulation. The liver protein alpha-tocopherol, transport protein(α-TTP) has been shown to be involved in this process. The sequence of α-TTP has been reported and the protein exhibits specificity for the RRR-α-tocopherol form as compared to the other isomers and forms of vitamin E. Another small molecular weight protein has been reported to be present in various tissues; however, the sequence or the role of this protein remains unidentified.

Recently, a protein was identified from humans and bovine as having specificity for the RRR-forms of tocopherol (Stocker et al., 1999; Zimmer et al., 2000). The protein is 46 KDa in mass and has a characteristic CRAL-TRIO domain, a domain involved in binding to hydrophobic ligands. This protein was called tocopherol-associated protein (TAP-46). A more recent paper, however, identified the identical protein as having a role in enhancing cholesterol biosynthesis by promoting the conversion of squalene to lanosterol and called the protein supernatant protein factor (SPF) (Shibata et al., 2001).

The prior art is lacking in means of inhibiting undesirable or uncontrollable cell proliferation in a wide variety of pathophysiological conditions while having no to little effect on normal cells. The present invention fulfills this long-standing need and desire in the art.

### SUMMARY OF THE INVENTION

Whether tocopherol-associated protein (TAP/SPF) plays a role in the ability of vitamin E compounds to induce tumor cells to undergo cell death by apoptosis was examined. cDNA from normal and breast cancer cells were cloned, and the presence of at least two tocopherol associated proteins, the previously reported tocopherol associated protein (TAP-46), and a novel tocopherol associated protein referred to herein as tocopherol associated protein p38 (TAP-38) were demonstrated.

TAP-38 cDNA differs from TAP-46 in that there is a 76 nucleotide deletion followed by 90 nucleotide mismatch sequences, and then an insertion of a single nucleotide. Thus, TAP-38 protein differs from TAP-46 protein by 55 amino acids (25 amino acid deletion and 30 novel amino acids). Evidence is provided that both TAP-38 and TAP-46 plays a role in the ability of vitamin E compounds to inhibit tumor cell growth.

The present invention also discloses three deletion mutants of tocopherol-associated protein (TAP) using TAP-46 as template. cDNA was generated by PCR with specific deletions of pcDNA3-TAP-46. TAP-882 is a deletion mutant with 330 base pairs deleted from the N-terminal (SEQ ID NOs: 14, 15). TAP-681 is a deletion mutant with 531 base pairs deleted from the N-terminal (SEQ ID NOs: 16, 17). TAP-456 is a deletion mutant with 756 base pairs deleted from the N-terminal (SEQ ID NOs: 18, 19). The predicted molecular weights of TAP-882, TAP-681 and TAP-456 are approximately 33 kDa, 29 kDa and 17 kDa respectively.

Polyclonal tocopherol-associated protein antibodies were produced in rabbits. The immunogen consisted of the 16 amino acids from the C-terminus of tocopherol-associated protein attached to keyhole limpet hemocyanin (KLH), resulting in the following immunogen: (KASEEKMKQLGAGTPK-KLH, SEQ ID NO: 8). The immunogen was prepared in complete Freunds adjuvant and rabbits were injected subcutaneously. Antibodies to the C-terminus of tocopherol-associated protein recognizes all of the tocopherol-associated protein deletion mutants.

In summary, this invention relates to cDNA cloning and nucleotide sequencing of tocopherol associated protein (TAP-38) from human normal and breast cancer cells, and data showing TAP-38 as well as TAP-46 to have a role in the ability of novel tocopherol compounds to induce cancer cells to undergo growth arrest via inhibition of DNA synthesis, induction of cellular differentiation, and induction of apoptosis. The present invention also discloses several deletion mutants of TAP-46.

Other and further aspects, features, benefits, and advantages of the present invention will be apparent from the following description of the presently preferred embodiments of the invention given for the purpose of disclosure.

### BRIEF DESCRIPTION OF THE DRAWINGS

FIG. 1 shows the cDNA sequence of TAP-38 (SEQ ID NO: 1).

FIG. 2 shows the predicted amino acid sequence of TAP-38.

FIG. 3 shows cDNA sequence comparison between TAP-38 and TAP-46.

FIG. 4 shows a predicted amino acid sequence comparison of TAP-38 and TAP-46 proteins.

FIG. 5 shows a schematic illustrating the amino acid deletion and novel amino acids of TAP-38 proteins.

FIG. 6 shows PCR products of TAP isolated from different breast cancer cell lines. (HEMC, human epithelial mammary cells; M10A, immortalized but non-tumorigenic human mammary cells; MDA-MB-435 and MDA-MB-231, estrogen non-responsive human breast cancer cells; MCF-7, estrogen responsive human beast cancer cells.

FIG. 7 shows a schematic illustrating the process for generating pGFP, pTRE, pGST, pHIS, and pcDNA3 plasmids.

FIG. 8 shows the expression of HA-tagged TAP-38 protein and HA-tagged TAP-46 proteins in MCF-7 and MDA-MB-435 human breast cancer cells.

FIG. 9 shows enhanced apoptosis [above levels obtained with cells transiently transfected with vector only (vector control)] of human MDA-MB-435 breast cancer cells transiently transfected with TAP-38 and TAP-46 cDNA, followed by treatment with 20 micrograms/ml of compound #1 (Co#-1).

FIG. 10 shows that antisense oligomers to TAP (both TAP-38 and TAP-46) transiently transfected into human MDA-MB-435 cells block (in comparison to sense oligomer transfected cells) the ability of vitamin E succinate (VES) and compound #1 (Co#-1) to induce human MDA-MB-435 cells to undergo apoptosis. Apoptosis was determined by examination of nuclear condensation and fragmentation.

FIG. 11 shows the expression of green fluorescent protein (GFP) in the cytosol of human MDA-MB-435 cells transiently transfected with pGFP vector (control), GFP-TAP-38 cDNA, and GFP-TAP-46 cDNA.

FIG. 12 shows that MDA-MB-435 cells transiently transfected with antisense (A) oligomers to TAP-38 or TAP-46 exhibit reduced levels of apoptosis [in comparison to sense oligomers (S)] when treated with apoptotic inducing agents vitamin E succinate (VES) and compound #1 (Co#-1). Apoptosis was measured by PARP cleavage. PARP 116 kDA, intact protein; PARP-84 kDA, cleavage product. Furthermore, antisense oligomers to TAP inhibited the phosphorylation of transcription factor protein c-Jun (pc-Jun).

FIGS. 13A-B show tocopherol associated protein p46 (TAP-46) is important for tocopherol-based compounds (α-TEA, VES and δT3) to induce MDA-MB-435 human breast cancer cells to undergo cell death by apoptosis, and that TAP small-interfering RNA (siRNA) is an effective blocker of TAP-46 expression. FIG. 13A shows that levels of TAP protein in cell lysates of human MDA-MB-435 breast cancer cells transiently transfected with siRNA targeted to TAP blocked the expression of TAP protein in a time-dependent manner. GAPDH levels were used as lane controls and for determining relative densitometric analyses. Si denotes cells transfected with TAP siRNA; Co denotes SiPORT lipid control levels; TAP denotes TAP-46. FIG. 13B shows that MDA-MB-435 cells transiently transfected with TAP siRNA for 2, 4 and 6 days, and then cultured for 2 days in the presence of α-TEA, VES or δT3were inhibited from undergoing apoptosis.

FIG. 14 is a schematic illustrating the similarities and differences between TAP-46, TAP-38, TAP-681, TAP-456 and TAP-882. TAP-38 has a 25 amino acid deletion followed by 30 unique amino acids. Homology between TAP-46 and TAP-38 resumes at amino acid 74. The deletion mutants are different deletions of the CRAL-TRIO domain. A polyclonal antibody to the C-terminus peptide recognizes all of the tocopherol-associated proteins.

FIG. 15 shows a schematic illustrating the process for generating pGFP, pTRE, pGST, pHIS, and pcDNA3 plasmids for the 3 TAP-46 deletion mutants.

FIG. 16 shows expression of TAP-46, TAP-38, TAP-882 and TAP-681 in MDA-MB-435 and MCF-7 human breast cancer cells. The pcDNA3-HA-TAP-882 and pcDNA3-HA-TAP-681 were transiently transfected into MDA-MB-435 and MCF-7 cells to overexpress HA-TAP-882 and HA-TAP-681. pcDNA3-HA-TAP-46 and pcDNA3-HA-TAP38 were also used as positive controls. Total cellular extracts were prepared and subjected to western immunoblot analysis, using rabbit antibodies to TAP C-terminus peptide. (UT=Untransfected cells).

### DETAILED DESCRIPTION OF THE INVENTION

The present invention is directed to an isolated and purified DNA encoding a tocopherol associated protein p38 having the amino acid sequence of SEQ ID NO: 2. In one aspect, the DNA has the sequence shown in SEQ ID NO: 1.

The present invention is also directed to a vector comprising the DNA of claim 1 and regulatory elements necessary for expressing said DNA in a cell, wherein the DNA encodes a tocopherol associated protein p38 having the amino acid sequence shown in SEQ ID NO: 2. In one aspect, the vector is a plasmid. For example, the plasmid may be a tetracycline regulated plasmid. The plasmid may encode a tocopherol associated protein p38 comprising a protein tag selected from the group consisting of a HA tag, a GST tag, a HIS tag and a green fluorescent protein tag.

The present invention is also directed to a host cell transfected with a vector described herein. Representative host cells include bacterial cells, mammalian cells, plant cells, yeast cells and insect cells.

The present invention is also directed to an isolated and purified tocopherol associated protein p38 having the amino acid sequence shown in SEQ ID NO: 2.

The present invention is also directed to an antibody directed against the tocopherol associated protein p38 described herein.

The present invention is also directed to an isolated and purified DNA encoding a deletion mutant of tocopherol associated protein having an amino acid sequence selected from the group consisting of SEQ ID NOs: 15, 17 and 19. Representative. DNA sequence are shown in SEQ ID NOs: 14, 16 and 18.

1. The present invention is also directed to a vector comprising the DNA and regulatory elements necessary for expressing said DNA in a cell, wherein the DNA encodes a deletion mutant of tocopherol associated protein having an amino acid sequence selected from the group consisting of SEQ ID NOs: 15, 17 and 19. The vector may be a plasmid such as a tetracycline regulated plasmid. More particularly, the plasmid may encode a deletion mutant of tocopherol associated protein comprising a protein tag selected from the group consisting of a HA tag, a GST tag, a HIS tag and a green fluorescent protein tag.

The present invention is also directed to an isolated and purified deletion mutant of tocopherol associated protein having an amino acid sequence selected from the group consisting of SEQ ID NOs: 15, 17 and 19.

The present invention is also directed to a mutated tocopherol associated protein p38, wherein said protein has a mutation that enhances biological function, said mutation selected from the group consisting of a mutation to the ligand binding domain, a mutation to the transactivation domain, a mutation to the nuclear localization domain, a mutation to the sequence specific DNA binding domain, a mutation to the non-sequence specific DNA binding domain, a mutation to the dimerization or tetramerization domain, and a mutation to a phosphorylation and dephosphrylation site.

The present invention is also directed to a method for the treatment of cell proliferative diseases comprising the step of administering to an animal a pharmacologically effective dose of the vector described above or a vector comprising a DNA that encodes a tocopherol associated protein p46 having the amino acid sequence shown in SEQ ID NO: 4. The animal treated may be a human or non-human. Representative cell proliferative diseases are neoplastic diseases and non-neoplastic disorders. Representative neoplastic disease are ovarian cancer, cervical cancer, endometrial cancer, bladder cancer, lung cancer, breast cancer, testicular cancer, prostate cancer, gliomas, fibrosarcomas, retinoblastomas, melanomas, soft tissue sarcomas, ostersarcomas, leukemias, colon cancer, carcinoma of the kidney, pancreatic cancer, basal cell carcinoma and squamous cell carcinoma. Representative non-neoplastic disease is selected from the group consisting of psoriasis, benign proliferative skin diseases, ichthyosis, papilloma, restinosis, scleroderma, hemangioma, leukoplakia, viral diseases, autoimmune disorders and autoimmune diseases. Representative autoimmune diseases are selected from the group consisting of autoimmune thyroiditis, multiple sclerosis, myasthenia gravis, systemic lupus erythematosus, dermatitis herpetiformis, celiac disease, and rheumatoid arthritis. A representative viral disease is caused by human immunodeficiency virus. Representative autoimmune disorders are selected from the group consisting of inflammatory processes involved in cardiovascular plaque formation, ultraviolet radiation induced skin damage, and disorders involving an immune component.

In a preferred aspect of this method, the vector is administered in the form of an aerosolized liposome. The liposome may comprise dilauroylphosphatidylcholine. This method may be used to inhibit tumor cell metastases.

In another preferred aspect of this method, the method further comprising the step of administering an anti-cancer drug to said animal, wherein said anti-cancer drug is administered at a time selected from the group consisting of before the administration of said vector, after the administration of said vector and concurrently with the administration of said vector. Representative anti-cancer drugs include 9-nitrocamptothecin, paclitaxel, doxorubicin, 9-nitrocamptothecin, 5-fluorouracil, mitoxantrone, vincristine, cisplatin, epoposide, tocotecan, tamoxifen, and carboplatin. In one aspect, the anti-cancer drug is administered in the form of an aerosolized liposome.

The present invention is also directed to an aerosolized liposome composition comprising a vector that encodes a tocopherol associated protein having an amino acid sequence selected from the group consisting of SEQ ID NOs: 2, 4, 15, 17 and 19. This liposome composition may comprise dilauroylphosphatidylcholine. This liposome composition may further comprise about 5% to 7.5% carbon dioxide. In one aspect, the liposome composition comprises polyethylenimine nitrogen and DNA phosphate at a ratio (nitrogen:phosphate) from about 5:1 to about 20:1.

The following definitions are given for facilitating understanding of the inventions disclosed herein. Any terms not specifically defined should be interpreted according to the common meaning of the term in the art.

As used herein, the terms “tocopherol associated protein p38 (TAP-38) cDNA and protein” and “tocopherol associated protein p46 (TAP-46) cDNA and protein” and “TAP-38 and TAP-46 antitumor functions” shall include the expression and analyses of TAP-38 and TAP-46 and constructs in vitro and in vivo.

As used herein, the term “individual” shall refer to animals and humans.

The term “biologically inhibiting” or “inhibition” of the growth of proliferating cells shall include partial or total growth inhibition and also is meant to include decreases in the rate of proliferation or growth of the cells. The biologically inhibitory dose of the composition of the present invention may be determined by assessing the effects of the test element on target malignant or abnormally proliferating cell growth in tissue culture, tumor growth in animals and cell culture or any other method known to those of ordinary skill in the art.

The term “induction of programmed cell death or apoptosis” shall include partial or total cell death with cells exhibiting established morphological and biochemical apoptotic characteristics. The dose of the composition of the present invention that induces apoptosis may be determined by assessing the effects of the test element on target malignant or abnormally proliferating cell growth in tissue culture, tumor growth in animals and cell culture or any other method known to those of ordinary skill in the art.

“Induction of cell cycle arrest” shall include growth arrest due to treated cells being blocked in GO/G1 or G2/M cell cycle phase. The dose of the composition of the present invention that induces cell cycle arrest may be determined by assessing the effects of the test element on target malignant or abnormally proliferating cell growth in tissue culture, tumor growth in animals and cell culture or any other method known to those of ordinary skill in the art.

“Induction of cellular differentiation” shall include growth arrest due to treated cells being induced to undergo cellular differentiation as defined by established morphological and biochemical differentiation characterization, a stage in which cellular proliferation does not occur. The dose of the composition of the present invention that induces cellular differentiation may be determined by assessing the effects of the test element on target malignant or abnormally proliferating cell growth in tissue culture, tumor growth in animals and cell culture or any other method known to those of ordinary skill in the art.

“Growth inhibitory concentration (IC₅₀)” or “effective concentration (EC₅₀)” shall include the effective therapeutic dose of a compound or composition for controlling cancer growth, i.e., by blocking 50% cancer growth via DNA synthesis inhibition, cellular differentiation, cell cycle blockage and/or cell death.

The term “inhibition of metastases” shall include partial or total inhibition of tumor cell migration from the primary site to other organs. The biological level of the composition of the present invention that enhances inhibition of metastasis by tocopherol based compounds may be determined by assessing the effects of the test element on target malignant or abnormally proliferating cell growth in tissue culture, tumor growth in animals and cell culture or any other method known to those of ordinary skill in the art.

The term “inhibition of angiogenesis” shall include partial or total inhibition of tumor blood vessel formation or reduction in blood carrying capacity of blood vessels supplying blood to tumors.

The present invention is directed toward the design and effective use of novel agents that can specifically target cancer cells and either down-regulate growth stimulatory signals, up-regulate growth inhibitory signals, down-regulate survival signals and/or up-regulate death signals. More specifically, this invention creates and characterizes novel agents (tocopherol associated protein p38) that activate growth inhibitory factors, trigger death signaling pathways and inhibit DNA synthesis.

The pharmacodynamically designed compounds of the present invention have an improved therapeutic index and are potent inhibitors of cancer cell growth, i.e., they demonstrate high antitumor activity with minimal side effects. These compounds, which cannot be readily degraded because there are no known etherases in mammals, may be used in the treatment of cancers and disorders involving excess cell proliferation, as well as for cells that accumulate in numbers due to suppressed cell killing mechanisms.

The compounds of the present invention inhibit cancer cell growth by induction of cell differentiation, induction of apoptosis and DNA synthesis arrest. Induction of apoptosis and, by extension, inhibition of tumor growth, by these compounds is mediated via modulation of the transforming growth factor-beta (TGF-β), Fas/Fas ligand, and certain mitogen-activated protein kinases (MAPK) signaling pathways, or, in the case of some tocotrienols, is expected to involve these pathways. Induction of apoptosis via other pathways, such as ceramide production, is not excluded. These growth inhibitory properties allow these compounds to be used in the treatment of proliferative diseases, including cancers of different cell types and lineages, non-neoplastic hyperproliferative diseases, and disorders with defects in apoptotic signaling pathways. Several of the compounds of the present invention are both strong inducers of apoptosis and strong inhibitors of DNA synthesis arrest of tumor cells representing different cellular lineages.

The methods of the present invention may be used to treat any animal. Most preferably, the methods of the present invention are useful in humans.

Stable and transient transfections, infections, or aerosol liposome method for delivery of TAP-38 or TAP-46, separately or in combination with other anticancer agents, may be used to treat neoplastic diseases and non-neoplastic diseases. Representative examples of neoplastic diseases are ovarian cancer, cervical cancer, endometrial cancer, bladder cancer, lung cancer, cervical cancer, breast cancer, prostate cancer, testicular cancer, gliomas, fibrosarcomas, retinoblastomas, melanomas, soft tissue sarcomas, osteosarcomas, colon cancer, carcinoma of the kidney, pancreatic cancer, basal cell carcinoma, and squamous cell carcinoma. Representative examples of non-neoplastic diseases include psoriasis, benign proliferative skin diseases, ichthyosis, papilloma, restinosis, scleroderma and hemangioma, and leukoplakia.

Methods of the present invention may be used to treat non-neoplastic diseases that develop due to failure of selected cells to undergo normal programmed cell death or apoptosis. Representative examples of diseases and disorders that occur due to the failure of cells to die are autoimmune diseases. Autoimmune diseases are characterized by immune cell destruction of self cells, tissues and organs. A representative group of autoimmune diseases includes autoimmune thyroiditis, multiple sclerosis, myasthenia gravis, systemic lupus erythematosus, dermatitis herpetiformis, celiac disease, and rheumatoid arthritis. This invention is not limited to autoimmunity, but includes all disorders having an immune component, such as the inflammatory process involved in cardiovascular plaque formation, or ultra violet radiation induced skin damage.

Methods of the present invention may also be used to treat disorders and diseases caused by viral infections, e.g. infection of human immunodeficiency viruses (HIV). Since the expression of TAP-38 or TAP-46 by tumor cells will likely render the cells more responsive to tocopherol based apoptotic inducing agents, this invention has the capacity to impact signal transduction of any type of external cellular signal such as cytokines, viruses, bacteria, toxins, heavy metals, etc.

Generally, to achieve pharmacologically efficacious cell killing and anti-proliferative effects, these compounds and analogs thereof may be administered in any therapeutically effective dose. Preferably, the structurally modified tocopherols and tocotrienols and analogs are administered in a dose of from about 0.1 mg/kg to about 100 mg/kg. More preferably, the structurally modified tocopherols and tocotrienols and analogs are administered in a dose of from about 1 mg/kg to about 10 mg/kg.

Administration of the compounds and compositions of the present invention may be by liposome/aerosol, topical, intraocular, parenteral, oral, intranasal, intravenous, intramuscular, subcutaneous, or any other suitable means. The dosage administered is dependent upon the age, clinical stage and extent of the disease or genetic predisposition of the individual, location, weight, kind of concurrent treatment, if any, and nature of the pathological or malignant condition. The effective delivery system useful in the method of the present invention may be employed in such forms as liposomal aerosol, capsules, tablets, liquid solutions, suspensions, or elixirs, for oral administration, or sterile liquid forms such as solutions, suspensions or emulsions. For topical use it may be employed in such forms as ointments, creams or sprays. Any inert carrier is preferably used in combination with suitable solubilizing agents, such as saline, or phosphate-buffered saline, or any such carrier in which the compounds used in the method of the present invention have suitable solubility properties.

In summary, there are a wide variety of pathological cancerous and noncancerous cell proliferative conditions and cell accumulations due to absence of normal cellular death for which the compositions and methods of the present invention will provide therapeutic benefits. These pathological conditions may occur in almost all cell types capable of abnormal cell proliferation or defective in programmed cell death mechanisms. Among the cell types which exhibit pathological or abnormal growth or abnormal death are fibroblasts, vascular endothelial cells and epithelial cells. It can be seen from the following experiments that the methods of the present invention are useful in treating local or disseminated pathological conditions in all or almost all organ and tissue systems of individuals.

In one aspect, the present invention is directed to isolated and purified DNAs encoding tocopherol associated protein p38 or deletion mutants of tocopherol associated protein. In general, the tocopherol associated protein p38 is encoded by DNA having the sequence of SEQ ID NO: 1, whereas the deletion mutants TAP-882, TAP-681 and TAP-456 are encoded by DNA having the sequences of SEQ ID NOs: 14, 16 and 18. The present invention also encompasses purified tocopherol associated protein p38 (SEQ ID NO: 2) and deletion mutants TAP-882, TAP-681 and TAP-456 (SEQ ID NOs: 15, 17 and 19).

In another aspect, the present invention provides vectors comprising the DNAs of the present invention and host cells comprising said vectors. Vectors of the invention include, but are not limited to, plasmid vectors and viral vectors. Preferred viral vectors can be derived from retroviruses, adenovirus, adeno-associated virus, SV40 virus, or herpes viruses. Preferably, the vector comprises a protein tag such as a HA-tag, a green fluorescent protein tag, a GST tag or a HIS tag. Use of green fluorescent protein tag permits one to determine if the tocopherol associated protein is regulated (translocated from cytosol to nucleus) by different forms of vitamin E. Use of GST tag permits analyses of phosphorylation status of tocopherol associated protein. Use of HIS tag permits the production and purification of high levels of tocopherol associated protein to be used for amino acid sequence analyses or vitamin E binding activity assays. In another embodiment, the vector is a tetracycline regulated plasmid comprising a doxocycline inducible tocopherol associated protein. This vector is useful for transfecting and selecting cell lines stably expressing tocopherol associated protein. Such cells can be used to examine the contributions of varying levels of tocopherol associated protein to the anti-tumor properties of vitamin E compounds.

In yet another aspect, the present invention is directed to an antibody directed against the tocopherol associated protein p38 of the present invention. Preferably, the antibody is a monoclonal antibody.

In another aspect, the present invention is directed to a mutated tocopherol associated protein p38, wherein said protein has a mutation that enhances biological function. Representative mutations include mutation to the ligand binding domain, mutation. to the transactivation domain, mutation to the nuclear localization domain, mutation to the sequence specific DNA binding domain, mutation to the non-sequence specific DNA binding domain, mutation to the dimerization or tetramerization domain, and mutation to a phosphorylation and dephosphrylation site.

In still yet another aspect, the present invention is directed to a method for the treatment of cell proliferative diseases comprising the step of administering to an animal a pharmacologically effective dose of a vector encoding tocopherol associated protein p38 (SEQ ID NO: 2) or tocopherol associated protein p46 (SEQ ID NO: 4). This method can be used to treat a human or non-human animal. Generally, this method may be used to treat a neoplastic disease or a non-neoplastic disorder. Representative neoplastic diseases and non-neoplastic disorders have been described above.

In a preferred embodiment of this treatment method, the vector is administered in the form of an aerosolized liposome. A representative liposome is formulated with dilauroyl-phosphatidylcholine and the aerosol may comprise about 5% to 7.5% carbon dioxide. More particularly, the aerosol may have a ratio of polyethylenimine nitrogen to DNA phosphate (nitrogen:phosphate) from about 5:1 to about 20:1. Generally, this method may be used to inhibit tumor cell growth by apoptosis, DNA synthesis arrest, cell cycle arrest, cellular differentiation or tumor cell metastases.

In another preferred embodiment of this treatment method, the method may further comprise the step of administering an anti-cancer compound before or after administering the vector. Representative anti-cancer drugs include 9-nitrocamptothecin, paclitaxel, doxorubicin, 5 -fluorouracil, mitoxantrone, vincristine, cisplatin, epoposide, tocotecan, tamoxifen, and carboplatin. The anti-cancer drug is preferably administered in the form of an aerosolized liposome. Optionally, the vector and the anti-cancer drug are administered concurrently in the form of an aerosolized liposome.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Cloning of Tocopherol Associated Protein p38 (TAP-38)

The coding area of the cDNA for human TAP was amplified by RT-PCR using total RNA from MDA-MB-43 5 and MCF-7 human breast cancer cell lines. The total RNA was extracted using RNeasy Mini Kit (Cat# 74104, Qiagen) following company instructions. The TAP oligonucleotide primers were synthesized based on the published TAP sequence (Accession # NM_{—}012429) with sense oligomer primer (5′-ATG AGC GGC AGA GTC GGC GAT-3′, SEQ ID NO: 5) and antisense oligomer primer (5′-TTA TTT CGG GGT GCC TGC CCC CA-3′, SEQ ID NO: 6) (Integrated DNA Technologies, Inc IDT). Five μg total RNA was used with random primer (Cat# 48190-011 lot# 1088038 GIBCOBRL). Total RNA was denaturated at 65° C. for 5 minutes, reverse transcribed at 42° C. for 50° C. mim and inactivated at 70° C. for 15 minutes. Five μl of RT-PCR product was used for PCR with 40 cycles of 94° C. for 30 s, 70° C. for 1 minute and 72° C. for 1 minute.

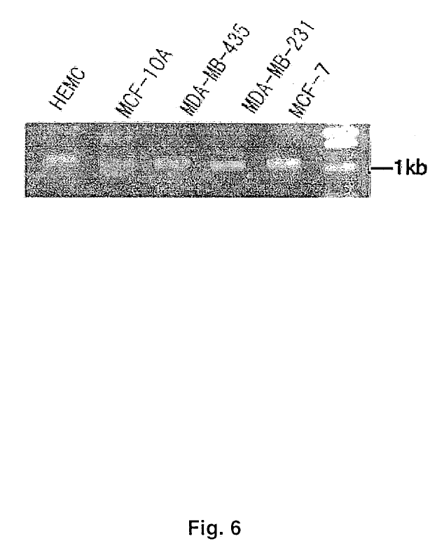

The about 1.2 kD PCR product (FIG. 6) was purified with QIAquick Gel Extraction Kit (Cat# 28704, Qiagen) and subcloned into the pGEM-T vector (Cat# A3610, Promega) after an A-tailing procedure following the company instructions. The construct was transformed into JM109 competent cells (Cat# A3610, Promega) using heat shock. Clones were sequenced using M13 forward and reverse oligomer primers (Integrated DNA Technologies, Inc IDT).

### EXAMPLE 2

cDNA Sequence Comparison Between TAP-38 And TAP-46

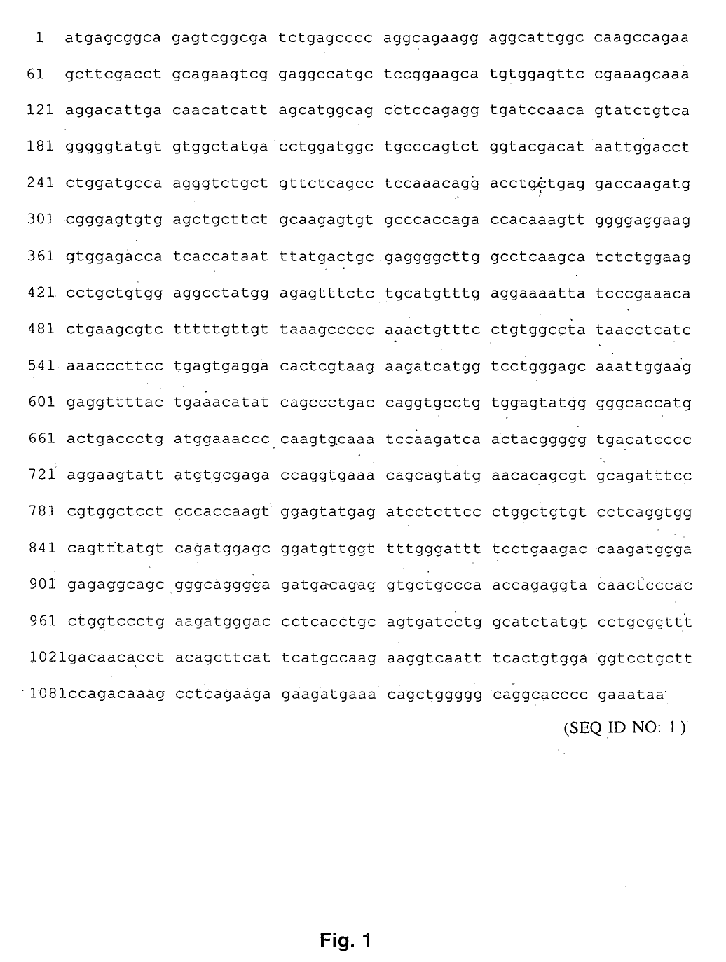

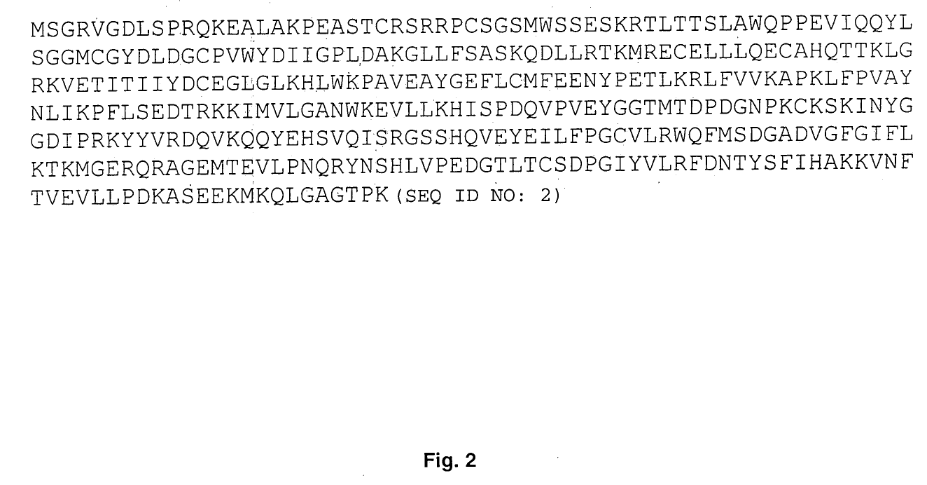

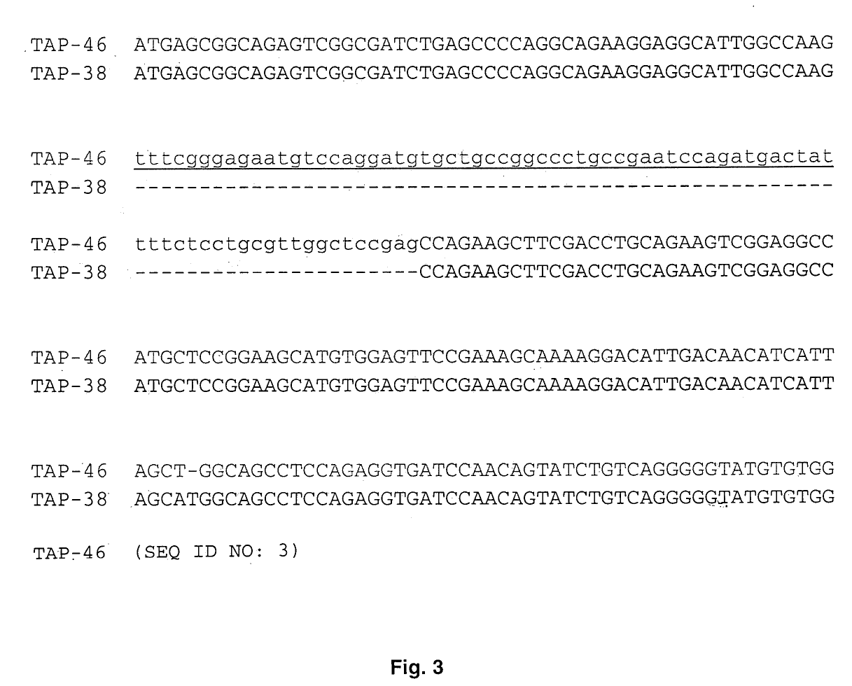

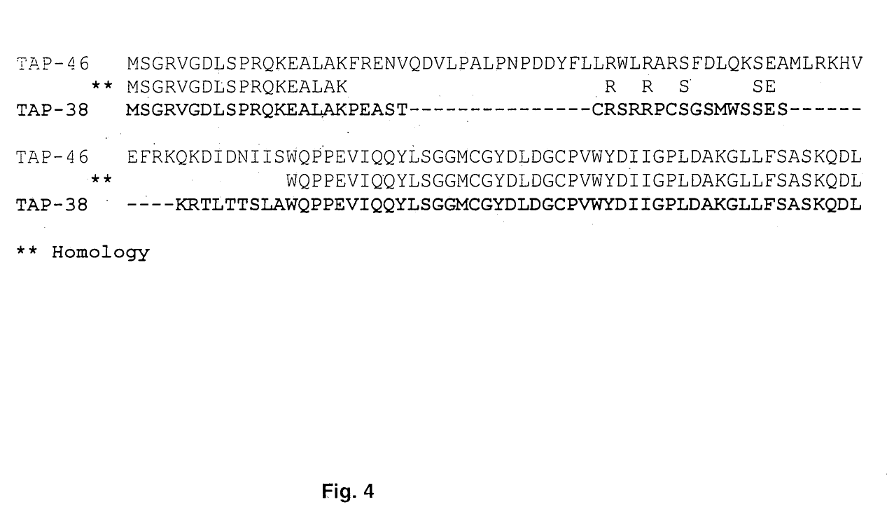

FIG. 1 shows a cDNA sequence comparison of TAP-38 with TAP-46. TAP-38 cDNA has a deletion starting at nucleotide 55 and continuing to nucleotide 131, resulting in a 76 base nucleotide deletion. There is a deletion of 25 amino acids and a disruption of the tocopherol associated protein triplets following nucleotide 131 and extending to nucleotide position 222. There is a single base nucleotide insertion at position 223. Thus, nucleotides 132 to 222 (90 nucleotides) code for novel TAP-38 amino acids (30 amino acids). TAP-38 nucleotides 224 to 1,137 exhibit 100% homology to TAP-46 nucleotides.

Consequently, TAP-38 protein is 25 amino acids shorter than TAP-46 (403 minus 25=378 amino acids), and further differs from TAP-46 by 30 additional amino acids. The 25 amino acid deletion occurs in the N-terminal domain of tocopherol associated protein, a region the function of which remains to be determined. TAP-38's novel 30 amino acids extends into the CRAL-TRIO domain of tocopherol associated protein by 10 amino acids. This domain has homology to TTP, retinal binding protein, SEC 14, PTN 9, and rat secretory protein 45.

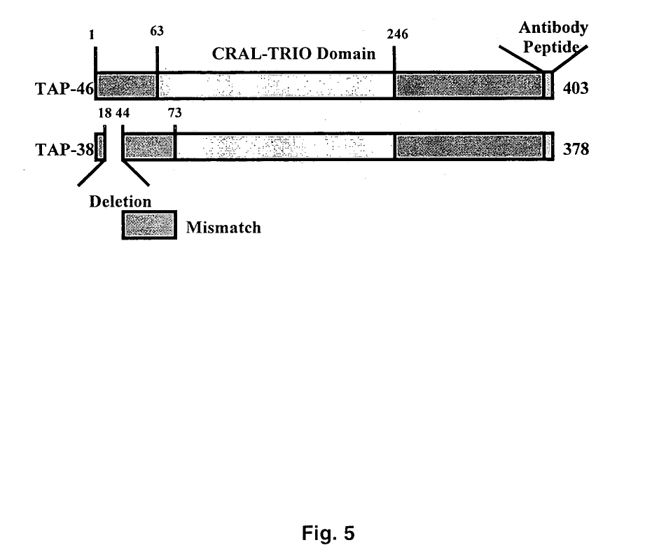

FIG. 5 is a schematic diagram of TAP-38 protein showing the position of the 25 amino acid deletion (amino acids 19-43) and the 30 novel amino acids (amino acids 44-73) in relation to TAP-46. With the exception of the 25 amino acid deletion and the 30 novel amino acids, Tap-38 exhibits 100% homology to other regions of TAP-46.

### EXAMPLE 3

Cloning of Tagged TAP-38 And TAP-461

For protein expression of 46 kDa and 38 kDa tocopherol associated protein a construct containing a HA-tag on the N-terminal site was designed. The sense primer for the PCR encoded an EcoRI restrict enzyme cutting site, starting codon and HA residue, and tocopherol associated protein sequence from 4-21 bases (5′-CGC GAA TTC ATG TAT GAT GTT CCT GAT TAT GCT AGC CTC AGC GGC AGA GTC GGC GAT, SEQ ID NO: 7), and the antisense primer contained a stop codon of tocopherol associated protein and BamHI restriction enzyme cutting site. The RT-PCR and PCR conditions were the same as described above. PCR products from MCF-7 and MDA-MB-435 cells were cloned into pGEM vectors. Three clones from each cell lines were sequenced using M13 forward and reverse oligomer primers (Integrated DNA Technologies, Inc IDT) as described above.

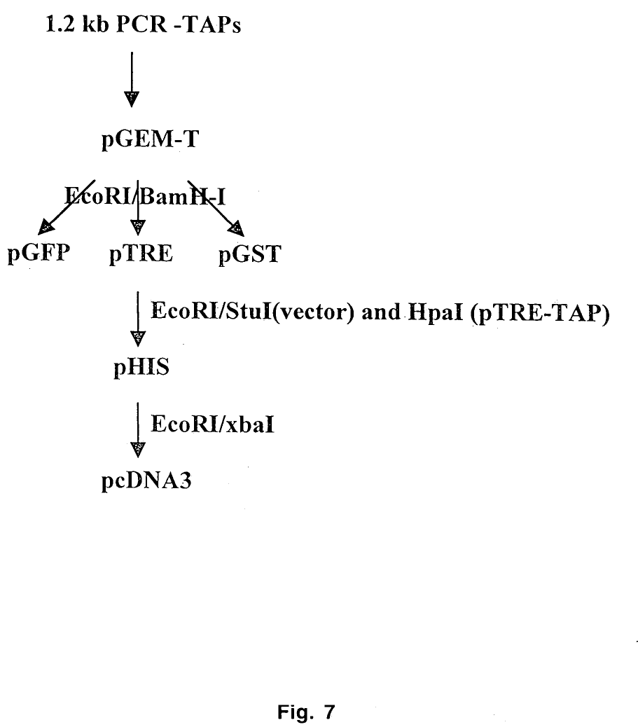

To generate different plasmids, the 1.2 kb PCR-TAP product was subcloned into the pGEM-T vector. Next, EcoRI and BamH-1 endnucleases were used to generate plasmids containing pGFP, pTRE, and pGST. The pTRE construct was used to generate plasmids containing pHIS (using endonucleases EcoRI/StuI (vector) and Hpal (pTRE-TAP), and plasmid containing pcDNA3 (using EcoRI/xbal endonucleases) (FIG. 7).

### EXAMPLE 4

Expression of TAP-38 And TAP-46

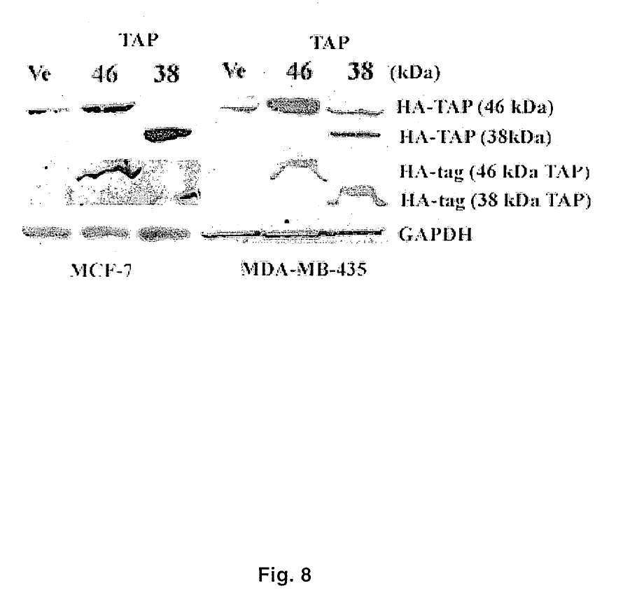

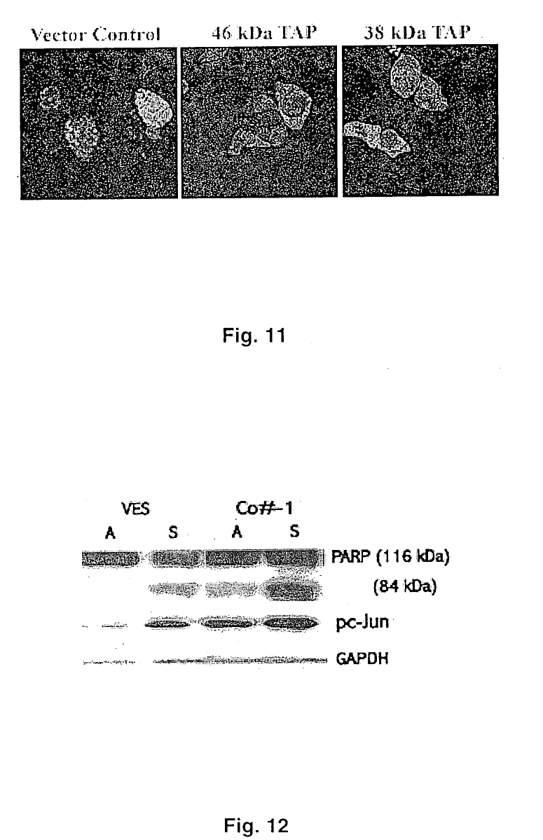

MCF-7 and MDA-MB-435 cells were stably transfected with pTRE-HA-TAP-38 and TAP-46 vectors, or transiently transfected with pcDNA-3 HA-TAP-38 and HA-TAP-46 vectors. Positive clones (three each) expressing HA-tagged TAP-38 and TAP-46 were selected by screening, using western blot with antibodies to HA-tag and antibodies to TAP C-terminus peptide (FIG. 8). FIG. 11 shows that green fluorescent protein (GFP)-tagged TAP-38 and TAP-46 were expressed and localized in the cytosol of transfected cancer cells.

### EXAMPLE 5

Role of TAP-38 or TAP-46 In The Induction of Apoptosis

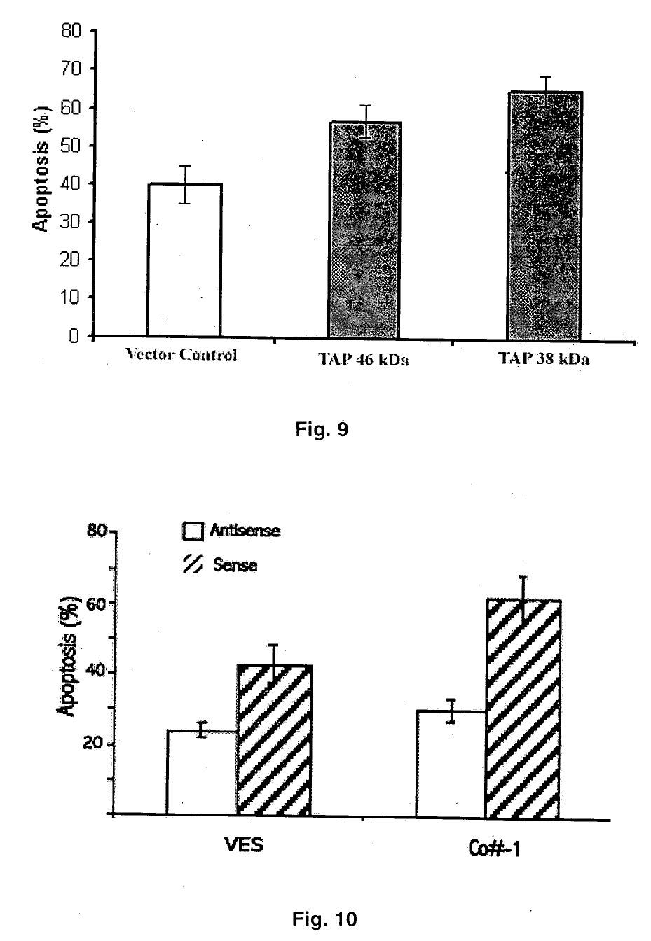

Transient transfection of MDA-MB-435 human breast cancer cells with either TAP-38 or TAP-46 enhanced the ability of tocopherol compound #1 [2,5,7,8-tetramethyl-(2R-(4R,8R,12-trimethyltridecycl) chroman-6-yloxy) acetic acid] to induce apoptosis (FIG. 9).

Transient transfection of MDA-MB-435 cells with antisense oligomers to the N-terminal region of tocopherol-associated protein (interferes with TAP-38 as well as TAP-46, transcription) blocked the ability of tocopherol compounds, vitamin E succinate and compound #1, to induce apoptosis, showing that TAP-38 and TAP-46 are involved in the ability of vitamin E compounds to inhibit tumor cell growth (FIG. 10).

Moreover, antisense oligomers to tocopherol associated protein blocked the cleavage of poly ADP-ribose polymerase (PARP) and phosphorylation of c-jun when the cells were treated with vitamin E succinate and compound #1 (FIG. 12).

### EXAMPLE 6

TAP-46 Blocking Experiments Using siRNA

MDA-MD-435 cells at 13.5×10⁶/55 cm² cell culture dish (100 mm×20 mm; catalog # 430293, Corning Inc., Corning, N.Y.) were transiently transfected with in vitro transcribed TAP siRNA (Silencer siRNA Construction Kit, Ambion) using siPORT Lipid following company instructions (catalog # 4505, Ambion, Austin, Tex.). Cells were incubated overnight in culture media, then washed two times with non-supplemented regular MEM media. Next, the cells were incubated overnight in the presence of siRNA/siPORT Lipid (10 mM/16 μl or 16 μl siPORT lipid only as control) in 5 ml of serum free media (OPTI-MEMI, catalog # 31985-070, Gibco). SiRNA/siPORT Lipid complex was generated by two steps: 1) 16 μl of siPORT lipid was incubated with 60 μl of OPTI-MEMI media for 30 minutes at room temperature; and 2) 60 μl of siPORT Lipid in OPTI-MEMI was then incubated for 20 minutes with 500 μl of diluted TAP siRNA (5 μl of 10 μM TAP siRNA in 500 μl of OPTI-MEMI media) or 500 μl media as control.

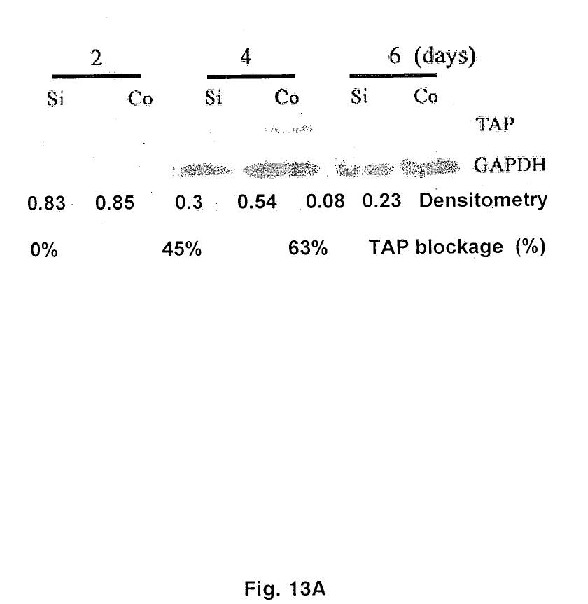

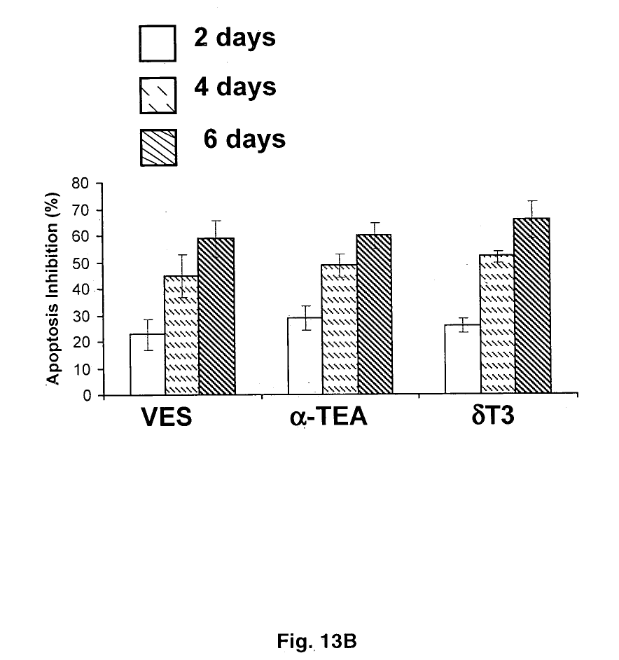

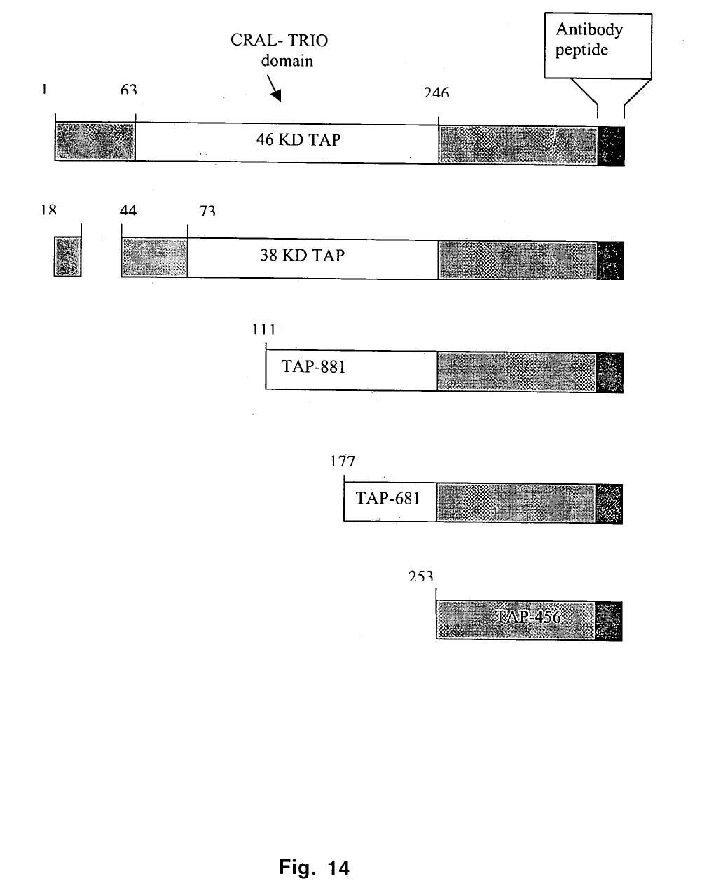

The transfected cells were then split into 12 wells at 1.5×10⁵ cells/well for apoptosis analysis and at 3.5×10⁶ cells/55 cm² dish for Western immunoblot analyses. The cells in 55 cm² dishes were incubated for 8 hours with culture media followed by treatment with 20 μg/ml of α-TEA in 2% serum for 15 hours. Cells were collected, fractionated, and the lysates were analyzed for TAP protein levels by Western immunoblotting (FIG. 13A). The cells plated in 12-well plates were incubated overnight in culture media followed by treatment with 20 μg/ml of α-TEA, 20 μg/ml of VES, and 5 μg/ml of RRR-δ-tocotrienol (dt3) and cultured for 2 days. Apoptosis was evaluated by DAPI staining (FIG. 13B). The results shown in FIGS. 13A-B demonstrate that tocopherol-associated protein p46 (TAP-46) is important for tocopherol-based compounds (including α-TEA, VES, and δT3) to induce MDA-MB-435 human breast cancer cells to undergo cell death by apoptosis.

FIG. 13A shows that levels of TAP protein in cell lysates of human MDA-435 breast cancer cells transiently transfected with small-interfering RNA (siRNA) targeted to tocopherol-associated protein blocked the expression of TAP protein in a time-dependent manner. Transient transfection of tocopherol-associated protein siRNA into MDA-MB-435 cells for 2, 4, and 6 days inhibited the expression of tocopherol-associated protein by 0%, 45%, and 63%, respectively. These data show that siRNA is an effective blocker for TAP-46 expression.

FIG. 13B shows that MDA-MB-435 breast cancer cells transiently transfected with tocopherol-associated protein siRNA for 2, 4, and 6 days, and then cultured for two days in the presence of VES, α-TEA or δT3 were inhibited from undergoing apoptosis. For example, cells transiently transfected with tocopherol-associated protein siRNA for 6 days and then treated with α-TEA, VES, or δT3 were inhibited from induction of apoptosis by approximately 60% in comparison to control cells cultured with the three compounds for two days.

### EXAMPLE 7

In Vivo Potential For Human Cancer Cells

The compositions of the present invention may be used as therapeutic agents. In vivo studies of tumor growth and metastasis can be conducted in well recognized animal models or in immune compromised animals such as nude mice transplanted ectopically or orthotopically with human tumor cells. Inhibition of growth of human tumor cells transplanted into immune compromised mice provide pre-clinical data for clinical trials. In vivo studies can be performed on the non-estrogen responsive MDA-MB-435 human breast cancer model, or a murine syngenic 66cl.4-GFP mammary cancer model.

MDA-MB-435 Breast Cancer Model

Pathogen free MDA-MB-435 human breast cancer cells stably transfected with a marker protein (green fluorescence protein, GFP) are grown as a solid tumor in immune compromised nude mice. One million tumor cells are orthotopically injected into the mammary fat pad or ectopically injected near the 4th and 5th nipples of female nude mice. When tumors reach a size of 1 mm, daily treatments with TAP-38 or TAP-46 plus tocophrol-based compounds exhibiting apoptosis-inducing properties are initiated. Tumor-growth, metastasis, and death of treated and control animals are determined. Tumor growth is measured by caliper evaluations of tumor size. At the time of sacrifice, tumors are removed, measured for volume, and used for histochemical examination. Organs such as spleen, lymph nodes, lungs, and bone marrow, are examined for metastatic cells by histochemical staining of tissue sections for expression of the marker green fluorescence protein.

Murine Syngeneic 66cl.4-GFP Mammary Cancer Model

Pathogen free 66cl.4-GFP mammary cancer cells of Balb/c origin (100,000 to 200,000) are injected near the 4th and 5th nipples of female Balb/c mice. Treatments are as described above. Tumor metastases to lungs occur in 100% of the mice. Tumor growth, metastasis, and death of the animals are determined. Tumor growth is measured by caliper evaluations of tumor size. At the time of sacrifice, tumors are removed, measured for volume, and used for histdchemical examination. Organs such as spleen, lymph nodes, lungs, and bone marrow, are examined for metastatic cells by histochemical staining of tissue sections for expression of the marker green fluorescence protein.

### EXAMPLE 8

Aerosol Liposome Administration of TAP-38 or TAP-46

TAP-38 or TAP-46 cDNA plasmid can be administered by infection, transfection, or by aerosol/liposomal preparation. The aerosol method is given here as an example of a method of delivery. Aerosol liposome/TAP38 or TAP-46 plasmid DNA preparation, in combination with tocopherol-based apoptosis-inducing agents (or in combination with other chemotherapeutic agents) can be administered to any animal, including humans.

A method of aerosol delivery is illustrated using mice as a test animal. The liposome/TAP38 or TAP-46 plasmid DNA preparation and tocopherol-based apoptosis-inducing compounds (with and without other chemotherapeutic agents) is administered to tumor bearing and non-tumor bearing Balb/c mice in a sealed plastic cage. An air compressor (EZ-Air PM 15F, Precision Medical) producing 10 L/min airflow is used with an Aero Mist nebulizer (CIS-US, Inc. Bedford, Mass.) to generate aerosol particles. The preparations are reconstituted by bringing the liposomes to room temperature before adding enough distilled water to bring the final volume to 5 mls. The solution is allowed to swell at room temperature for 30 minutes with periodic inversion and then added to the nebulizer. The nebulizer is connected via accordian tubing (1 cm inside diameter) to an entry in one end of the cage. Aerosol is discharged through an opening at the opposite end of the cage. For safety, nebulization is done in a hood. Aerosol is administered to the mice in a closed container cage until all treatment is gone (approximately 30 minutes for delivery of total volume of 5 mls).

### EXAMPLE 9

Cloning of TAP-46 Deletion Mutants

The pcDNA3-TAP-46 construct was used as a template for construction of mutant tocopherol associated proteins. TAP-882 is a deletion mutant with 330 base pairs deleted from the N-terminal. TAP-681 is a deletion mutant with 531 base pairs deleted from the N-terminal. TAP-456 is a deletion mutant with 756 base pairs deleted from the N-terminal (FIG. 14). The predicted molecular weights of TAP-882, TAP-681 and TAP-456 are approximately 33 kDa, 29 kDa and 17 kDa respectively.

The sense primers for PCR encoded an EcoRI restriction enzyme cutting site (GAA TTC), starting codon (ATG) and sequence for an HA tag (TAT GAT GTT CCT GAT TAT GCT AGC CTC, SEQ ID NO: 9) and TAP sequence. The sense primer for the deletion mutant TAP-882 had the sequence 5′-CGC GAA TTC ATG TAT GAT GTT CCT GAT TAT GCT AGC CTC CTG CTG TTC TCA GCC TCC AA-3″ (SEQ ID NO: 10). For the HA-TAP-681 insert, primer sequence used was 5′-CGC GAA TTC ATG TAT GAT GTT CCT GAT TAT GCT AGC CTC TTT GAG GAA AAT TAT CCC GA-3″ (SEQ ID NO: 11). For the HA-TAP-456 insert, the primer sequence used was 5′-CGC GAA TTC ATG TAT GAT GTT CCT GAT TAT GCT AGC CTC AAG TGC AAA TCC AAG ATC AA-3″ (SEQ ID NO: 12). The antisense primer was common for the three mutants and contained a BamHI restriction enzyme cutting site (GGA TCC), stop codon (TTA) and the following antisense sequence (5′TTT CGG GGT GCC TGC CCC CAG-3′, SEQ ID NO: 13). (Integrated DNA technologies). The resulting PCR products with sizes of 882 bp, 681 bp and 456 bp for the three mutants were purified using a QIAquick PCR purification Kit (Qiagen) and subcloned into the pGEM-T bacterial vector (Promega) after an A-tailing procedure as per the company's recommendations. The construct was transformed into DH5α subcloning efficiency cells (Invitrogen). Three clones from each transformation were sequenced using the T7 and SP6 sequencing primers (Integrated DNA technologies).

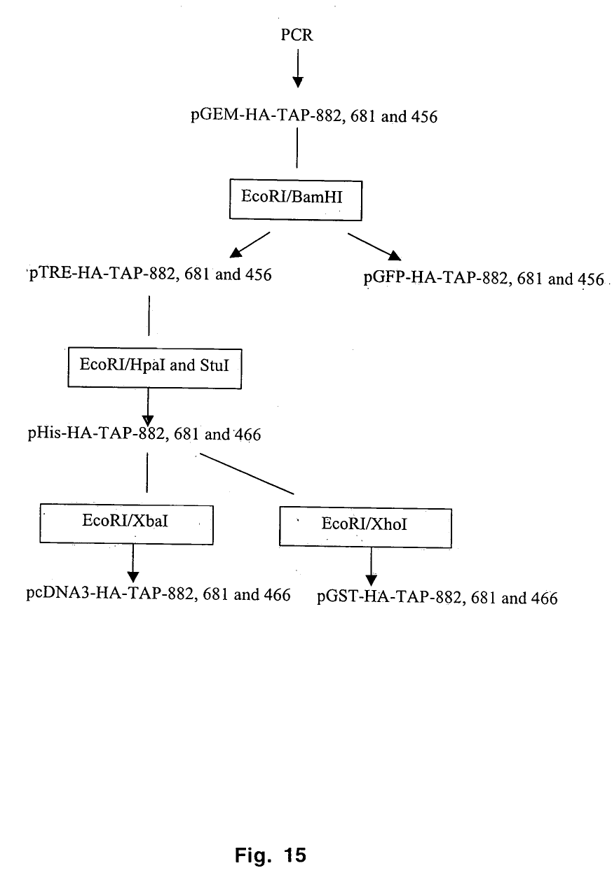

FIG. 15 shows a schematic illustrating the process for generating pGFP, pTRE, pGST, pHIS, and pcDNA3 plasmids for the 3 TAP-46 deletion mutants. To create the construct of pTRE-HA-TAP-882, pTRE-HA-TAP-681, and pTRE-HA-TAP-456, HA-TAP-882, HA-TAP-681, and HA-TAP-456 were subcloned from pGEM-HA-TAP-882, pGEM-HA-TAP-681 and pGEM-HA-TAP-456 vectors into the pTRE vector (Clontech, Palo Alto, Calif.) using EcoRI/BamHI restriction enzymes.

To create the constructs pGFP-HA-TAP-882, pGFP-HA-TAP-681, and pGFP-HA-TAP-456, pGEM-HA-TAP-882, pGEM-HA-TAP-681, and pGEM-HA-TAP-456 vectors were digested by EcoRI/BamHI to get HA-TAP-882, HA-TAP-681, and HA-TAP-456 which were subcloned into pGFP-2 vector (Novagen).

To create the constructs pHis-HA-TAP-882, pHis-HA-TAP-681, and pHis-HA-TAP-456, pTRE-HA-TAP-882, pTRE- HA-TAP-681 and pTRE-HA-TAP-456 were digested first with EcoRI, then purified using QIAquick gel extraction kit (Qiagen) and then digested with HpaI to get EcoRI/HpaI digested fragments. Inserts carrying the EcoRI/HpaI restriction enzyme cutting sites were subcloned into the expression vector pPROEXTM (Life technologies) which was digested with EcoRI/StuI restriction enzymes. The resulting pHis-HA-TAP-882, pHis-HA-TAP-681, and pHis-HA-TAP-456 constructs contains 6 His residues that would bind to a Ni-NTA affinity column.

To construct pcDNA3-HA-TAP-882, pcDNA3-HA-TAP-681, and pcDNA3-HA-TAP-456, HA-TAP-882, HA-TAP-681, and HA-TAP-456 were subcloned from pHis-HA-TAP-882, pHis-HA-TAP-681 and pHis-HA-TAP-456 vectors into the pcDNA3 vector (Invitrogen) using EcoRI/XbaI restriction enzymes.

To creat pGST-HA-TAP-882, pGST-HA-TAP-681, and pGST-HA-TAP-456, HA-TAP-882, HA-TAP-681, and HA-TAP-456 were subcloned from pHis-HA-TAP-882, pHis-HA-TAP-681 and pHis-HA-TAP-456 vectors into the pGST expression vector pET-41a (+) (Novagen) using EcoRI/XhoI restriction enzymes.

### EXAMPLE 10

Transient Transfection of TAP-46 Deletion Mutants

MDA-MB-435 and MCF-7 human breast cancer cells were allowed to adhere overnight, washed twice with serum-free medium (MEM) and then incubated for 6-7 hours with 0.5 ml of Opti-MEM® serum free medium (Life technologies) containing 100 μl of plasmid/LipofectAMINETM Plus® reagent complex in 12 well plates or in 3 ml of Opti-MEM® serum free medium containing 700 μl of plasmid/LipofectAMINETM Plus® reagent complex in T-25 flasks. The plasmid/LipofectAMINETM Plus® reagent complexes were prepared by mixing 0.7 μg of plasmid DNA/50 μl of serum free media with 4 μl of Plus reagent with 2 μl of LipofectAMINETM® reagent/50 μl of serum free media followed by 15 minutes of incubation.

The cells were plated at 5×10⁶ cells per T-75 flask for Western immunoblotting or 1.5×10⁵ cells/well in 12-well plates for apoptosis analysis. For apoptosis studies, the cells were treated with various concentration of RRR-α-tocopheryl succinate or α-TEA and examined as described above.

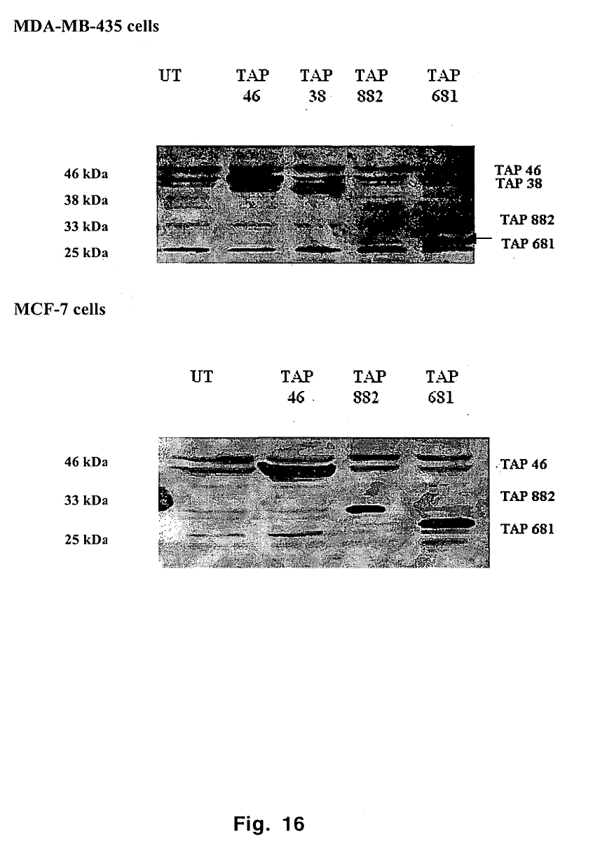

For western blot analysis, cells lysates were collected by centrifugation, and 100 ug/lane of protein were loaded onto SDS-PAGE gel. Proteins were separated by electrophoresis and transferred to nitrocellulose membranes. Following blocking, the membranes were reacted with 1:1000 of primary rabbit antibody to human TAP, washed, reacted with horseradish peroxidase conjugated goat anti-rabbit IgG secondary antibody at 1:2000 dilution for 30 minutes. Protein levels were detected by enhanced chemoluminescence. Results from one of these experiments were shown in FIG. 16.

### EXAMPLE 11

Purification of His-Tagged TAP-46 Deletion Mutants

For inducible exogenous protein expression, subcloning efficiency DH5α strain of E. coli (Invitrogen) was transformed with pHis-HA-TAP-882, pHis-HA-TAP-681 and pHis-HA-TAP-456. The cells were grown in a liquid Lennox L Broth Base (LB) culture media (BIO, Vista, Calif.) to a spectrophotometrically determined density A 590 of 0.5-1.0 units, and the expression of His-HA-tocopherol-associated protein was induced by 0.6 mM isopropylthiogalactopyranoside (IPTG) for 3 hours. The cells were harvested by centrifugation at 6000×g for 10 minutes and resuspended in 4 volumes of lysis buffer (50 mM Tris-HCL, pH 8.5 at 4° C., 5 mM 2-mercaptoethanol, 1 mM freshly prepared PMSF). To lyse the bacteria, they were sonicated using autotune series high intensity ultrasonic processor (Sonics and Materials INC, Newtown, Conn.) for 8 bursts, each burst lasting 30 seconds with 30 seconds gap between each burst. This sonication procedure produced approximately 90 percent lysis as determined by spectrophotometric measurement of density. The lysate was centrifuged at 6000×g for 30 minutes to remove cell debris.

The supernatant containing solubilized His-HA-TAP-882, His-HA-TAP-681, and His-HA-TAP-456 proteins was purified using affinity chromatography with Ni-NTA resin (Life technologies, Cat # 10711-018). Buffer A (20 mM Tris-HCL (pH 8.5 at 4° C.), 100 mM KCl, 5 mM 2-mercaptoethanol, 10% glycerol, 20 mM imidazole) was used to prequilibriate the Ni-NTA resin to 50%. Two ml 50% slurry Ni-NTA resin was mixed with 15 ml bacterial lysis supernatant continuously for 1 hour at 4° C. with constant rotation. Next the resin was centrifuged for 2 minutes at 6000×g and the supernatant was discarded. Then the resin was washed thrice using 1 ml Buffer A. His-HA-TAP-882, His-HA-TAP-681, and His-HA-TAP-456 proteins were eluted from Ni-NTA resin using Elution Buffer (20 mM Tris-HCL (pH 8.5 at 4° C.), 100 mM KCl, 5 mM 2-mercaptoethanol, 10% glycerol, and 100 mM imidazole). Purity of the eluted tocopherol-associated protein deletion mutants was determined using Western immunoblot employing rabbit anti-tocopherol-associated protein polyclonal antibody.

The following references are cited herein.

Stocker et al., Identification of a novel cytosolic tocopherol-binding protein: structure, specificity, and tissue distribution. IUBMB Life 48:49-55 (1999).

Zimmer et al., A novel human tocopherol-associated protein: cloning, in vitro expression, and characterization. J. Biol. Chem. 275:25672-25680 (2000).

Shibata et al., Supernatant protein factor, which stimulates the conversion of squalene to lanosterol, is a cytosolic squalene transfer protein and enhances cholesterol biosynthesis. Proc. Natl. Acad. Sci. U.S.A. 98:2244-2249 (2001).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. An isolated and purified DNA encoding a tocopherol associated protein p38 having the amino acid sequence of SEQ ID NO: 2.

2. The DNA of claim 1, wherein said DNA has the sequence shown in SEQ ID NO: 1.

3. A vector comprising the DNA of claim 1 and regulatory elements necessary for expressing said DNA in a cell, wherein said DNA encodes a tocopherol associated protein p38 having the amino acid sequence shown in SEQ ID NO: 2.

4. The vector of claim 3, wherein said vector is a plasmid.

5. The vector of claim 4, wherein said plasmid is a tetracycline regulated plasmid.

6. The vector of claim 4, wherein said plasmid encodes a tocopherol associated protein p38 comprising a protein tag selected from the group consisting of a HA tag, a GST tag, a HIS tag and a green fluorescent protein tag.

7. A host cell transfected with the vector of claim 3.

8. The host cell of claim 7, wherein said cell is selected from the group consisting of bacterial cells, mammalian cells, plant cells, yeast cells and insect cells.

9. An isolated and purified tocopherol associated protein p38 having the amino acid sequence shown in SEQ ID NO: 2.

10. An antibody directed against the tocopherol associated protein p38 of claim 9.

11. An isolated and purified DNA encoding a deletion mutant of tocopherol associated protein having an amino acid sequence selected from the group consisting of SEQ ID NOs: 15, 17 and 19.

12. The DNA of claim 11, wherein said DNA has a sequence selected from the group consisting of SEQ ID NOs: 14, 16 and 18.

13. A vector comprising the DNA of claim 11 and regulatory elements necessary for expressing said DNA in a cell, wherein said DNA encodes a deletion mutant of tocopherol associated protein having an amino acid sequence selected from the group consisting of SEQ ID NOs: 15, 17 and 19.

14. The vector of claim 13, wherein said vector is a plasmid.

15. The vector of claim 14, wherein said plasmid is a tetracycline regulated plasmid.

16. The vector of claim 14, wherein said plasmid encodes a deletion mutant of tocopherol associated protein comprising a protein tag selected from the group consisting of a HA tag, a GST tag, a HIS tag and a green fluorescent protein tag.

17. A host cell transfected with the vector of claim 13.

18. The host cell, of claim 17, wherein said cell is selected from the group consisting of bacterial cells, mammalian cells, plant cells, yeast cells and insect cells.

19. An isolated and purified deletion mutant of tocopherol associated protein having an amino acid sequence selected from the group consisting of SEQ ID NOs: 15, 17 and 19.

20. A mutated tocopherol associated protein p38, wherein said protein has a mutation that enhances biological function, said mutation selected from the group consisting of a mutation to the ligand binding domain, a mutation to the transactivation domain, a mutation to the nuclear localization domain, a mutation to the sequence specific DNA binding domain, a mutation to the non-sequence specific DNA binding domain, a mutation to the dimerization or tetramerization domain, and a mutation to a phosphorylation and dephosphrylation site.

21. A method for the treatment of cell proliferative diseases comprising the step of administering to an animal a pharmacologically effective dose of the vector of claim 3 or a vector comprising a DNA that encodes a tocopherol associated protein p46 having the amino acid sequence shown in SEQ ID NO: 4.

22. The method of claims 21, wherein said animal is a human or non-human.

23. The method of claims 21, wherein said cell proliferative disease is selected from the group consisting of neoplastic diseases and non-neoplastic disorders.

24. The method of claim 23, wherein said neoplastic disease is selected from the group consisting of ovarian cancer, cervical cancer, endometrial cancer, bladder cancer, lung cancer, breast cancer, testicular cancer, prostate cancer, gliomas, fibrosarcomas, retinoblastomas, melanomas, soft tissue sarcomas, ostersarcomas, leukemias, colon cancer, carcinoma of the kidney, pancreatic cancer, basal cell carcinoma and squamous cell carcinoma.

25. The method of claim 23, wherein said non-neoplastic disease is selected from the group consisting of psoriasis, benign proliferative skin diseases, ichthyosis, papilloma, restinosis, scleroderma, hemangioma, leukoplakia, viral diseases, autoimmune disorders and autoimmune diseases.

26. The method of claim 25, wherein said autoimmune diseases are selected from the group consisting of autoimmune thyroiditis, multiple sclerosis, myasthenia gravis, systemic lupus erythematosus, dermatitis herpetiformis, celiac disease, and rheumatoid arthritis.

27. The method of claim 25, wherein said viral diseases is caused by human immunodeficiency virus.

28. The method of claim 25, wherein said autoimmune disorders are selected from the group consisting of inflammatory processes involved in cardiovascular plaque formation, ultraviolet radiation induced skin damage, and disorders involving an immune component.

29. The method of claim 21, wherein said vector is administered in the form of an aerosolized liposome.

30. The method of claim 29, wherein said liposome is dilauroylphosphatidylcholine.

31. The method of claim 21, wherein said method inhibits tumor cell metastases.

32. The method of claim 21, further comprising the step of administering an anti-cancer drug to said animal, wherein said anti-cancer drug is administered at a time selected from the group consisting of before the administration of said vector, after the administration of said vector and concurrently with the administration of said vector.

33. The method of claim 32, wherein said anti-cancer drug is selected from the group consisting of 9-nitrocamptothecin, paclitaxel, doxorubicin, 9-nitrocamptothecin, 5-fluorouracil, mitoxantrone, vincristine, cisplatin, epoposide, tocotecan, tamoxifen, and carboplatin.

34. The method of claim 32, wherein said anti-cancer drug is administered in the form of an aerosolized liposome.

35. An aerosolized liposome composition comprising a vector that encodes a tocopherol associated protein having an amino acid sequence selected from the group consisting of SEQ ID NOs: 2, 4, 15, 17 and 19.

36. The liposome composition of claim 35, wherein said liposome is dilauroylphosphatidylcholine.

37. The liposome composition of claim 35, wherein said composition comprises about 5% to 7.5% carbon dioxide.

38. The liposome composition of claim 35, wherein said composition comprises polyethylenimine nitrogen and DNA phosphate at a ratio (nitrogen:phosphate) from about 5:1 to about 20:1.
